(19) United States
(12) Patent Application Publication
(10) Pub. No.: US 2023/0176002 A1
(43) Pub. Date: Jun. 8, 2023

(54) HIGH SENSITIVITY METAL-COMPOSITE POROUS GRAPHENE OXIDE CAPACITIVE ORGANOPHOSPHATE SENSOR

(71) Applicant: B.G. NEGEV TECHNOLOGIES AND APPLICATIONS LTD., AT BEN-GURION UNIVERSITY, Beer Sheva (IL)

(72) Inventors: Raz JELINEK, Modi'in-Maccabim-Re'ut (IL); Nitzan SHAULOFF, Be'er Sheva (IL); Nagappa L. TERADAL, Belagavi (IN)

(21) Appl. No.: 17/923,877

(22) PCT Filed: May 6, 2021
(86) PCT No.: PCT/IL2021/050522

§ 371 Date: Nov. 7, 2022

(30) Foreign Application Priority Data
May 8, 2020 (IL) 274539

(51) Int. Cl. G01N 27/22; G01N 33/00

(52) U.S. Cl. CPC G01N 27/227; G01N 33/0047

(57) ABSTRACT

Provided herein a capacitive organophosphate vapor-detecting sensors, methods of manufacturing thereof, and sensing devices comprising same. The sensors comprise an electrode and metal-composite porous graphene oxide dielectric material, integrally formed on said electrode.

### BACKGROUND OF THE INVENTION

Organophosphates (OPs) are highly toxic, and their use as pesticides and chemical warfare agents pose significant health, environmental, and security risks. In particular, OPs exhibit long-term neurotoxic effects, as well as genotoxic and reproduction damages. Accordingly, varied technologies have been developed for detection and monitoring OPs. OP sensing in solution is generally carried out by small molecule-based probes. Such chemosensors usually undergo optical transformations occurring through chemical reactions with the target analytes. Electrochemical sensing of OPs has been also reported, relying on electrodeposition of ZrOCl₂ upon gold surfaces. Discrimination of biologically-relevant OPs was accomplished through multivalent detection using a hyperbranched polymer coupled to a fluorescent dye. Powder-based detection of OPs was carried out via a glove-embedded printable biosensor, possibly facilitating on-site detection of OP-based nerve-agent compounds.

Technologies for detection of OP vapors, however, have been limited, primarily due to insufficient selectivity and specificity or the sensing platforms. Implementing electrochemical OP vapor sensing via biological molecules has been actively pursued. The indirect detection of OPs through inhibition of enzymes acetylcholinesterase or butyrylcholinesterase have been studied. These systems, however, usually require relatively complicated multi-step protocols, highly controlled conditions, long-term storage stability, and because the enzymes are inhibited by many other chemicals (heavy metals, carbamates, etc.), the sensor selectivity is poor.

Transition metal ions have been employed as vehicles for OP vapor detection, specifically through formation of metal complexes in which OP residues constituted the ligands. OP sensing modes in metal-based systems have relied on modulations of metal complexes' colors, fluorescence, or electronic properties, endowing useful versatility to these systems. For example, detection of OP vapors has been achieved through ligand substitution in hybrid nanocomposites consisting of copper oxide nanowires coupled to single-walled carbon nanotubes, affecting electric current modulation. High sensitivity has been reported in sensing gases, particularly CO₂, using the resistive properties of cobalt-doped hydroxyapatite (Mahabole, M. P.; Mene, R. U.; Khairnar, R. S. Gas Sensing and Dielectric Studies on Cobalt Doped Hydroxyapatite Thick Films. Advanced Materials Letters 2013, 4 (1), 46-52). There have been limitations in such systems as practical sensing platforms due to slow sensor response times, high operating temperatures, complex synthetic schemes, significant signal variability which depended upon experimental conditions, and sophisticated instrumentation.

Capacitive vapor sensors, which operate via modulation of the capacitance by physical or chemical adsorption of volatile molecules onto the sensor material, are attractive due to their low response times, reproducibility, low power consumption, and ambient temperature applicability. Since capacitive sensors have no static power consumption, they are suitable for use in energy-constrained applications, such as low-power battery-operated systems and wireless sensor networks. An important advantage of capacitance-based gas sensing is the fact that detection properties are determined by dielectric modulation, generally exhibiting higher fidelity and sensitivity than charge effects which are dominant in resistance-based sensors. Some highly sensitive versatile capacitive vapor sensors have been described in U.S. Pat. No. 10,890,550, utilizing porous graphene oxide (pGO). The sensors detect with high precision the relative humidity over a very broad range, and also accurately determine with the concentration of ammonia in concentrations as low as between 1 and 70 ppm, as well as threshold concentrations of volatile organic compounds, such as ethanol, phenol, acetonitrile, and benzene, with very short response and recovery times.

There is a need in the art to provide a sensitive OP sensor with short response time, and preferably acceptable the recovery time.

### SUMMARY OF THE INVENTION

It has now been surprisingly found that capacitive sensors could be fabricated to detect organophosphates, with a very short response time, by incorporating into porous graphene oxide sensors some specific transition metal cations, particularly divalent transition metal cations of cobalt and of nickel, i.e. Co²⁺ and Ni²⁺. As demonstrated in the appended Examples, the sensors exhibit extraordinary sensitivity, displaying impressive 340 capacitance response upon interactions with threshold concentrations of organophosphate compounds (e.g. triethyl-phosphate), and up to 1000 capacitance response over the tested concentrations range. Without being bound by a particular theory it is believed that the extraordinary sensing properties are likely ascribed to structural reorganization of the pGO framework by the embedded metal ions, and subsequent substitution of the ligands within the metal complexes by the OP molecules, as supported by spectroscopic and microscopic analyses. The disclosed OP vapor sensors are easy to prepare, and their superior sensing properties may be employed in practical OP alert systems. As demonstrated in the appended Examples section below, the sensors were able to react to the presence of as little as 5 parts per million by volume (ppmv) of triethyl phosphate, and showed linearity throughout the tested range of up to 100 ppmv, with very little or no cross-reactivity to volatile organic compounds in comparable concentrations. This threshold concentration is much lower than warfare OP concentrations estimated to affect humans (e.g. sarin (GB) half-lethal exposure is about 17 ppmv/min, and tabun (GA) 30-61 ppmv/min, adapted from N Munro et al, 1994, Environmental Health Perspectives, 102:1, doi: 10.1289/ehp. 9410218), as well as for a popular agrochemical dichlorvos—the dangerous level is considered 22 ppmv.

Thus, in a first aspect, provided herein is a capacitive organophosphate vapor sensor, comprising porous graphene oxide which is composite with transition metal cations, adsorbed on an electrode. Preferably, the transition metal cations are selected from Co²⁺ and Ni²⁺. Further preferably, the sensor comprises at least one pair of electrodes and metal-composite porous graphene oxide, integrally formed on said at least one pair of electrodes. In a further aspect provided herein a method of manufacturing a capacitive organophosphate vapor sensor, the method comprising combining graphene oxide with transition metal cation source, e.g. a salt of a transition metal, to obtain metal-composite porous graphene oxide precursor. The method further comprises applying said precursor to at least one pair of electrodes, furnishing a precursor sensor assembly. The method further comprises expanding in situ said metal-composite porous graphene precursor, on said precursor sensor assembly, to obtain metal-composite porous graphene oxide integrally formed on said at least one pair of electrodes, e.g. a capacitive organophosphate vapor sensor. Preferably, the method comprises combining a dispersion comprising graphene oxide, e.g. an aqueous dispersion, and a transition metal cation source, e.g. a salt of a transition metal, to obtain metal-composite porous graphene oxide precursor liquid, applying said precursor liquid to at least one pair of electrodes, preferably for at least a 5-minute application period, and freeze-frying said metal-composite porous graphene oxide precursor liquid on said at least one pair of electrodes, to furnish a sensor according to the present invention.

Provided herein a capacitive sensor for detection of organophosphate vapors, said sensor comprising dielectric material integrally formed on a pair of electrodes, said dielectric material comprising transition-metal composite of porous graphene oxide. In the sensor, said composite comprises between 5 and 12 weight percent of a transition metal. Optionally, the sensor comprises between 7 and 9 weight percent of a transition metal. Usually, said transition metal in said composite is selected such that the dielectric constant of said dielectric material is above 150 F/m. Optionally, said dialectic constant is above 1000 F/m. In the sensor said transition metal in said composite is usually selected such that in the composite a ratio between the area under the Raman signal appearing at ˜1350 cm⁻¹ and the area under the Raman signal appearing at ˜1575 cm⁻¹ is between 1 and 1.9. The sensor is usually such that the dielectric material comprises between 8 and 25 weight percent of adsorbed water. Optionally, said dielectric material comprises between 14 and 22 weight percent of adsorbed water. For the sensor said transition metal is usually selected from the group consisting of cobalt, nickel, titanium, ruthenium, palladium, and zirconium. Optionally, said transition metal is present in a form of a cation. Optionally said transition metal is Co²⁺ or Ni²⁺. The sensor usually comprises said pair of electrodes which are interdigitated electrodes.

Further. Provided herein a sensing device for the detection of organophosphates vapors in the air, said device comprising a capacitive sensor according to any one of the preceding claims. The device optionally further comprises a temperature controlling unit. The device optionally further comprising a humidity compensation sensor. Optionally, said temperature controlling unit is in thermal connection with said capacitive sensor. In the device said capacitive sensor is being preferably conductively connected to an electrical circuit adapted to monitor the capacitance of the sensor. The device may further comprise an effector sub-circuit configured to produce a notification upon a change in the capacitance of said sensor, indicative of the presence of an organophosphate vapor. The notification may usually be in a form of an alarm sound, in a form of deflection of a pointer, or in a form of an electromagnetic signal. The may also comprise a plurality of said capacitive sensors in form of an array.

Further, provided herein a process of manufacturing a sensor as described above and generally herein, said process comprising providing a pair of electrode and integrally forming thereon a coating comprising metal-composite porous graphene oxide. The metal-composite porous graphene oxide preferably comprises cobalt or nickel. The wherein a weight ratio between said metal and said graphene oxide is usually between 5 and 12 weight percent. The process may further comprise providing a metal-composite graphene oxide precursor liquid, by combining in an aqueous medium a metal source and a graphene oxide dispersion. The metal precursor is usually an inorganic salt of said metal. The a weight ratio between said metal and said graphene oxide may usually be between 0.3:1 and 1:1, in said precursor liquid. The process may further comprise purifying said metal-composite porous graphene oxide precursor liquid, e.g. by separating said metal-composite graphene precursor and said aqueous medium, and resuspending said separated metal-composite porous graphene oxide precursor in water. The process further comprises applying said metal-composite porous graphene oxide precursor liquid onto said pair of electrodes. The applying is usually performed at a temperature ranging from 10° C. to 60° C. The applying is usually performed for an incubation time of at least 5 minutes. The incubation time may also be between 45 and 75 minutes. The process may further comprise freeze-drying said metal-composite porous graphene oxide precursor liquid on said electrode. The process is usually such that the amount of water in said precursor liquid after said incubation time and before said freeze-drying is between 25% and 40%.

### BRIEF DESCRIPTION OF THE DRAWINGS

FIG. 1 schematically demonstrates an experimental setup to test the performance of sensors in vapor-sensing applications.

FIG. 2 represents an electron scanning micrograph of Co²⁺-composite porous graphene oxide integrally formed on an interdigitated electrode.

FIG. 3 represents an EDS spectrum (energy dispersive x-ray spectroscopy) of Co²⁺—composite porous graphene oxide integrally formed on an interdigitated electrode.

FIG. 4 represents the XPS survey spectrum (x-ray photoelectron spectroscopy) of Co²⁺-composite porous graphene oxide integrally formed on an interdigitated electrode.

FIG. 5 represents the high-resolution O-1 s XPS spectrum of Co²⁺-composite porous graphene oxide integrally formed on an interdigitated electrode.

FIG. 6 represents RAMAN spectra of the metal-composite porous graphene oxide embodiments according to the invention and the comparative data.

FIG. 7 represents capacitance signals of an embodiment according to the invention, upon exposure to an organophosphate vapor and after the removal thereof with a concentration of 30 ppmv.

FIG. 8 represents capacitance signals of an embodiment according to the invention, upon repeated adsorption/desorption cycles.

FIG. 9 represents capacitance response in an embodiment according to the present invention, in form of dose-response curve in the range of 0-70 ppmv TEP concentration.

FIG. 10 represents raw capacitive response of pGO-Co²⁺ to TEP vapors between concentrations of 5-30 ppmv.

FIG. 11 represents a graph demonstrating stability and repeatability of capacitive response signals of pGO-Co²⁺ recorded upon adsorption and desorption of TEP at a concentration of 30 ppmv to triethyl-phosphate vapor over time.

FIG. 12 represents a graph demonstrating the capacitance response of pGO-Co²⁺ at different relative humidity values.

FIG. 13 represents a graph demonstrating the capacitive response of pGO-Co²⁺ to triethyl-phosphate vapor in air atmosphere at a concentration of 30 ppmv.

FIG. 14 represents a graph demonstrating the capacitive response of pGO-Co²⁺ as function of temperatures between 25° C. and 55° C.

FIG. 15 represents a graph demonstrating the capacitive response of pGO-Co²⁺ upon exposure to gas mixes comprising organophosphate vapors.

### DETAILED DESCRIPTION

Surprisingly, the inventors have now developed a new type of capacitive vapor sensor for detection of organophosphates, by incorporating transition metal cations into porous graphene oxide (pGO) produced through an in-situ process upon the electrode surface, whereby the sensor thus produced has the pGO immobilized on the electrode surface. Some specific transition metal cations are particularly useful for the present invention, such as divalent transition metal cations of cobalt and of nickel, i.e. Co²⁺ and Ni²⁺.

Thus, the sensors comprise metal-composite porous graphene oxide, particularly cobalt-composite porous graphene oxide, and nickel-composite porous graphene oxide. The sensor further comprises at least one pair of electrodes, between which the metal-composite porous graphene oxide is deposited as dielectric material, to produce a capacitor. The properties of the capacitor react to the changes in ambient conditions, most particularly with the presence of organophosphate compounds vapor. Organophosphate compounds may interact with the metal ions in the metal-composite porous graphene oxide (pGO-Mⁿ⁺) structure, e.g. by displacing water from coordinational bonds, thereby changing the dielectric constant and thus changing the capacitance. This change in capacitance can be readily detected by electrical means as known in the art. As demonstrated in the appended Examples, the capacitance change produced by exposing the sensor to organophosphate vapor, even to as low concentration as 5 parts per million by volume (ppmv), can produce a threefold change in the capacitance, and up to 700 capacitance response signal with the maximal tested concentration of the organophosphate vapor, and perhaps beyond. Therefore, the threshold detection concentration of various organophosphate vapors may be as low as 5 ppmv, but depending on the nature of the organophosphate and its molecular mass, and of course the capacitance response by the sensor, could be even lower. In order to refer to specific concentrations of vapors as presented herein, the concentrations unit of ppmv is used, indicating volumes of gaseous contaminant in one million of volumes of air/contaminant mixture. The conversion from ppmv to mass per volume units, e.g. mg/m³, is readily performed as known in the art, e.g. by expressing the volume of the gaseous contaminant through the mass using ideal gas law PV=nRT, wherein P is the pressure of the gas in kiloPascals (kPa), V is the volume of the gas in liters (L), n is the molar amount of the gas in moles, T is the absolute temperature in Kelvin (K), and R is the ideal gas constant, equal to 8.3144 L*kPa*mol−1*K⁻¹.

Thus, the dielectric material of the capacitor is a modified porous graphene oxide, as disclosed herein. Porous graphene oxide is a particular form of graphene oxide, which is usually characterized by significant spacing of sp2-carbon layers of graphene oxide, preferably by assembling them into a three-dimensional framework, or by creating holes in graphene sheets. Porous graphene oxide can be easily distinguished from non-porous graphene oxide by a number of properties, as known to a person skilled in the art, e.g. by the surface area measurement, and/or pore-size analysis.

Porous graphene oxide according to the invention is usually modified with transition metal ions. There are several useful properties of the transition metal ions according to the invention. The transition metal ions may be selected according to their ability to interact with graphene oxide without impairing its structural integrity. As demonstrated in the Examples section, a useful metal ion incorporated into porous graphene oxide structure, should demonstrate in RAMAN spectroscopy a ratio between the areas of the ID Raman peak (area under the Raman signal appearing at 1350 cm⁻¹) and IG peak (at around ˜1575 cm⁻¹), that is usually higher than 1, but lower than 1.9, e.g. between 1.1 and 1.7.

Further, the suitable metal ion should have sufficient coordination number to allow bonding of various ligands, e.g. water, apart from edge groups of graphene oxide. Thus, a suitable ion should produce a composite porous graphene oxide that could be characterized by a loss on drying at about 125° C. of over 8 weight percent, preferably over 10 weight percent. The loss on drying (i.e. the content of water) of the composite porous graphene oxide is thus usually between 8 and 25 weight percent, e.g. between 14 and 22 weight percent, or between 15 and 20 weight percent.

Further preferably, the metal ion should not decrease the dielectric constant of the composite porous graphene oxide to below 50 percent of that of unmodified porous graphene oxide prepared without the metal. Preferably, the dielectric constant of the composite porous graphene oxide is higher than the dielectric constant of the corresponding porous graphene oxide without the metal, and can be above 150 F/m, e.g. above 500 F/m, and currently preferably above 1000 F/m, e.g. between 150 and 5,000 F/m, preferably between 500 and 3,000 F/m, and further preferably between 1000 and 2500 F/m.

The transition metal ions are preferably divalent cations of transition metals. Further preferably, the ions are Co²⁺ and Ni²⁺, but may also include cations of zirconium, titanium, ruthenium, and palladium.

The weight/molar fraction of the metal in the composite porous graphene oxide may usually depend on the metal and the metal ion used. Generally, the amount of the metal in the composite may be such that it does not adversely affect the dielectric constant, yet allows sufficient metal to be present in the composite. The exemplary ranges of weight fractions for various metals may be between 3 and 20 weight percent, preferably between 5 and 12 percent, further preferably between 7 and 9 percent.

Porous graphene oxide composites are formed from graphene oxide composites precursors, preferably integrally formed in situ, e.g. on the electrodes. This term “integrally formed”, e.g. on the electrodes, unless the context dictates otherwise, should be construed such that pores are formed in graphene oxide material which has been applied to the electrodes, thus creating porous graphene oxide directly and integrally on the electrodes. The pores in graphene oxide may be created by various means, such as modification of the graphene oxide to allow of the modification groups intercalation between the graphene oxide sheets, but preferably the pores are created by a physical process. The physical processes may include expansion and freeze-drying; preferably metal-composite porous graphene oxide is freeze-dried from aqueous precursor slurry of metal-graphene oxide composite. Other processes to create porous graphene oxide are enumerated below.

To prepare a sensor, the metal-composite porous graphene oxide is adsorbed on the electrode surface, e.g. at least part of the available surface area of the electrode which is not necessarily the outer geometric surface area of the electrode. The electrode used to provide the sensor of the present invention can be any type of a pair of interdigitated electrodes (IDE), which can provide rapid response, low impedance, allowing for simple detection of impedance changes, e.g. via high current changes at constant voltage. As used herein, the terms “interdigitated electrode(s)” or “interdigitated microelectrode(s)” indicates at least two complementarily-shaped electrodes, wherein “branches” or “fingers” of each electrode are disposed in an alternating fashion. The two electrodes are not in a direct electric contact with one another, but can be connected into an electrical chain as capacitor. The IDE can comprise gold, silver, platinum, or indium tin oxide (ITO). Preferably, the IDE comprises gold.

The metal-composite porous graphene oxide is adsorbed onto the electrode surfaces by integrally forming on the electrode surfaces. This immobilization may be the result of either chemical or physical bonding between the GO sheets in a precursor or of the obtained pGO, and the electrode surface. This physical or chemical attachment or adsorption usually occurs in the first step of the electrode preparation process, as detailed below (i.e. drying of the GO modified electrode at room temperature). Without being bound to any specific theory, it is believed that the attachment of the GO and pGO to the electrode occurs through weak physical interaction between the functional groups (hydroxyl, epoxy, and carboxyl groups) of the graphene oxide and the metal electrode surface.

As used herein, the term “capacitive sensor” designates a sensor, which generates a signal responsive to the influence of what is being sensed (such as an analyte) upon an electric field. A capacitive sensor generally comprises at least one antenna electrode, to which is applied an oscillating electric signal and which thereupon emits an electric field into a region of space proximate to the antenna electrode, while the sensor is operating. The sensor comprises at least one sensing electrode—which may be identical with or different from transmitting antenna electrodes—at which the influence of the analyte on the electric field is detected.

According to one preferred embodiment of the present invention, at the tested concentrations of organophosphates, the response time of the sensors are lower than 50 seconds, and the recovery times are lower than 600 seconds. However, it should be borne in mind that recovery and response time ranges usually cannot be precisely and unequivocally defined for any sensor, as they depend particularly on the type of the analyte, e.g. the OP, and the metal-composite material used.

The capacitive sensors for detection of organophosphates may be produced by a method comprising combining graphene oxide with transition metal cation source, e.g. a salt of a transition metal, to obtain metal-composite porous graphene oxide precursor. The precursor may be purified from the unreacted/unadsorbed metal residues, e.g. by centrifugation and washing with water. The method further comprises applying said precursor to at least one pair of electrodes, e.g. IDE, furnishing a precursor sensor assembly. The method further comprises expanding in situ said metal-composite porous graphene precursor, on said precursor sensor assembly, to obtain metal-composite porous graphene oxide integrally formed on said at least one pair of electrodes, e.g. a capacitive organophosphate vapor sensor.

The in-situ creation of the pores within the GO attached to the electrode surface can be effected by a number of methods and processes known in the field, some of which are listed herein. These include, but are not limited to, hydrothermal processes, irradiation, polymerization, grafting, template based, annealing, electroplating deposition, oxidative coupling of primary amines, steam etching, expansion and freeze-drying. Some of the processes are described in U.S. Pat. No. 10,890,550, incorporated herein by reference.

Preferably, freeze-drying may be used to create pores by drying the solidified GO/water mixture under vacuum. The freeze drying may be performed as known in the art, and can be achieved in one step or in several steps. Alternatively, ammonium carbonate may be used in a precursor to create pores, by expulsion of gaseous ammonia or carbonate created by decomposition of the salt under heat. For example, the creating of the pores in the graphene oxide was obtained by heating a suspension of metal-composite graphene oxide and ammonium carbonate, which was drop casted onto the electrode, to obtain a porous graphene oxide film adsorbed on the electrode.

Preferably, the method of manufacturing the capacitive sensor for the detection of organophosphate vapors comprises the steps of combining a dispersion comprising graphene oxide, e.g. an aqueous dispersion, and a transition metal cation source, e.g. a salt of a transition metal, to obtain metal-composite porous graphene oxide precursor liquid; applying said precursor liquid to at least one pair of electrodes, preferably for at least a 5-minute application period; and freeze-frying said metal-composite porous graphene oxide precursor liquid on said at least one pair of electrodes, to furnish a sensor according to the present invention.

Thus, metal-composite porous graphene oxide precursor liquid may be prepared by combining, in an aqueous medium, graphene oxide and a salt of transition metal. The salt is preferably an inorganic salt, e.g. a halide, preferably chloride, a sulfate, acetate and nitrate. The salt is preferably soluble in water to a sufficient extent to allow incorporation into graphene oxide. The salt may usually be provided in excess, to ensure complete saturation of the binding sites on graphene oxide. The molar concentration of the salt in the precursor liquid may usually be in the range of between 2 and 10 mM, preferably between 4 and 6 mM. The weight ratio between the transition metal and the graphene oxide in the precursor liquid may usually be between 0.3:1 and 1:1, preferably between 0.4:1 and 0.7:1 weight percent. The graphene oxide dispersion with the excess of transition metal salt may be stirred for a time interval sufficient for the incorporation of the metal, e.g. between 5 minutes and 6 hours, preferably between 1 and 3 hours. The resultant dispersion may be then purified from the excess of the transition metal salt, e.g. by centrifugation and decantation of the supernatant, followed by re-dispersion of the pellet in water, preferably in deionized water. The process may be repeated as needed, until all excess of transition metal salt is removed.

The metal-composite porous graphene oxide precursor liquid is then contacted with at least one pair of electrodes, and left to allow adsorption of the graphene oxide—metal composite onto the surface of the electrodes, i.e. applying the precursor liquid onto the electrodes. The time interval to allow adsorption may vary between 5 minutes and 2 hours, such as between 15 minutes and 90 minutes preferably between 45 and 75 minutes. The particular time interval may be determined according to the residual water content, as described further herein. During this time interval, it is believed without being bound by any particular theory, that the [hydrated] polar groups of graphene oxide create contact with the electrodes, which in turn allows the dielectric material of the capacitor, i.e. metal-composite porous graphene oxide, to be formed integrally on the electrodes. During the application time interval the precursor liquid may be allowed to evaporate partially, e.g. to retain sufficient amount of liquid to enable expansion during the subsequent lyophilization step. Preferably, the amount of residual water in the precursor liquid by the end of the application step may be between 23 and 50% (by weight), preferably between 25 and 40% (by weight). It is noted that the inventors have shown that pre-dried metal-composite porous graphene oxide which was not applied in-situ to the electrode, will not attach to the electrode surface. The in-situ adsorption of the GO on the electrode surface is preferably done at temperatures ranging from about 10° C. to about 60° C. However, the drying can be effected at temperatures that are even higher than 60° C., thereby lowering the adsorbing time.

The remaining liquid may be removed from the precursor liquid, e.g. by freeze-drying of the sensor, as known in the art. Generally, the sensor is frozen to a temperature sufficiently low to maintain the precursor liquid frozen for the time required for the pressure to decrease to below the that of triple point of water, e.g. below about 4.58 mm Hg, to effect sublimation of water from the precursor liquid.

The organophosphate vapor sensor, as described generally herein, may be used in an organophosphate vapor sensing device.

The OP-vapor sensing device may comprise at least one transition metal-composite porous graphene oxide capacitive sensor, conductively connected to an electrical circuit adapted to monitor the capacitance of the sensor. In this connection, the sensor may be connected as a regular capacitor, conductively connected to a circuit, which comprises an effector sub-circuit. The effector sub-circuit is configured such that upon a change in the capacitance of the sensor, indicative of the presence of an organophosphate vapor, the effector sub-circuit produces a notification. The notification may be in form of an alarm sound, in form of deflection of a pointer, in form of providing a signal to an external device, e.g. an electromagnetic signal, or in any other form known in the art. Preferably, the sensing device comprises a plurality of OP-vapor sensing capacitive sensors, e.g. in form of an array; for the simplicity, unless the context clearly dictates otherwise, the singular term “sensor” is used herein to describe also the arrays of plurality of sensors.

The sensor may be directly accessible to the ambience, to effect the sensing of organophosphate vapors. However, it may be advantageous to provide means for supplying the external air to a sensor located in a controlled environment, e.g. via an air supply path. The air supply path may include other units ensuring proper functioning of the sensing device, e.g. particle filters, temperature controlling means, isolation means to limit or restrict the access to the sensor from the ambience, and other parts as known in the art.

The OP sensing device may further include a temperature controlling unit. As it can be seen from the appended examples, the capacitance change of the sensor may change significantly responsive to a temperature change. Therefore it may be advantageous to maintain the sensor at a constant temperature. This may be carried out by any form known in the art, including but not limited to, by providing a Peltier heat pump, providing a heating coil, providing a cooling unit, or a combination of the means, in the vicinity of the sensor, and/or along the pathway of the sampled air. The temperature controlling unit may further comprise a thermometer or a thermocouple, i.e. a monitored temperature measuring device, to ensure the correct functioning of the temperature controlling unit. The temperature controlling unit may thus be in a thermal connection with the sensor, e.g. via the thermocouple monitoring the temperature of the sensor, thereby directly measuring the temperature thereof. The temperature controlling unit may also be in thermal connection with the air in the vicinity of or downstream to the sensor, thereby measuring the temperature thereof indirectly.

The OP sensing device may further include a humidity compensation sensor. As it can be seen from the appended examples, the capacitance change of the sensor may change to a certain extent responsive to the relative humidity change. Therefore it may be advantageous to include into the sensing device a humidity compensation sensor. The humidity-compensation sensor may be, e.g. one of the sensors disclosed in the U.S. Pat. No. 10,890,550. The response from the humidity compensation sensor may be modulated electrically to compensate for the humidity change in the sensing sensor, along the disclosed in the appended examples, or can be modulated digitally in a processing unit.

The OP sensing device may further include a purging assembly, to supply an inert gas to the sensor, to assist in recovery of the sensor, or a part of sensors in an array of sensors. The purging assembly may comprise a conduit for providing an inert gas to the vicinity of the sensor. The purging assembly may be electrically connectable to or digitally modifiable by the temperature controlling unit, e.g. to allow elevation of temperature to facilitate the recovery of the sensor.

The OP sensing device may further include a storage assembly, to provide optimal storage conditions for the sensor during storage. The storage assembly may comprise isolation means to block or to limit the access of external air to the sensor. The storage assembly may further comprise a dosing unit to provide periodically an inert gas to the sensor.

The term “sensor”, used interchangeably with the term “detector”, may particularly denote any device which may be used for the detection of an analyte. Examples for sensors which may be realized according to exemplary embodiments are organophosphate vapor sensors, humidity sensors, etc. As used herein, the term “analyte” used interchangeably with the term “target molecule” indicates a molecule whose presence, absence, or concentration one is interested in determining. The term “vapor sensor”, used interchangeably with the term “gas sensor”, refers to any device which may be used for the detection of an analyte comprising particles in the gas phase. For example, the vapor sensor may be used for the selective detection of a gas in a gas mixture. According to preferred embodiments of the invention, the analyte or target molecule is in a vapor form, and thus the sensor is a vapor sensor and detects the presence, absence, or concentration of vapor target molecules. For example, the sensor of the present invention may be used to determine whether or not the amount of organophosphate vapor in the sample exceeds a pre-determined level. The term “capacitive vapor sensor” usually refers to a capacitor having an electric characteristic which is modifiable by a sensor event, in the present case, a sensor that changes its dielectric properties, such as capacitance, in contact with the vapor target molecules.

The term “capacitor” refers to a device for storing electrostatic energy through the separation of electric charges of opposite signs. All capacitors share a common structure of a pair of parallel metallic electrodes or “plates” separated by a layer of dielectric material. The capacitor is “charged” by transferring electric charge from one electrode to the other under the action of an applied potential difference, thus establishing an electric field within the dielectric material. The dielectric material (also termed dielectric medium, dielectric core, or dielectric substance) of the capacitors of the present invention is the metal-composite porous graphene oxide (pGO-Mⁿ⁺), integrally formed in situ on the electrode surface, preferably using the process of the present invention.

As used herein the term “capacitance” is expressed by the equation C as a function of voltage=dQ/dV where C is capacitance measured in farads, Q is the quantity of charge in coulombs, and V is the applied voltage in volts. Depending upon its magnitude, capacitance can be expressed in farads, F, microfarads, μF=10⁻⁶ F, or picofarads pF=10⁻¹² F. As can be seen in the figures hereinbelow, the capacitance response is often provided as C₀/C_{gas}, where Co is the capacitance baseline and C_{gas} is the capacitance after passing the gas analyte. The term “humidity” refers to water vapor and may in particular denote an absolute humidity, a mixing ratio or a humidity ratio, a relative humidity, and/or a specific humidity of a gas-liquid mixture such as an air-water mixture. The term “humidity sensor” may particularly denote any device which may be used for the detection of water. For example, the humidity sensor may be used to detect to measure humidity, i.e. an amount of a water vapor in the air. As employed herein, the term “dynamic range” means the ratio or difference between the smallest and largest possible values of a changeable quantity (e.g., without limitation, amplitude; magnitude).

Unless otherwise defined, all technical and scientific terms used herein have the same meaning as is commonly understood by one of skill in the art to which this invention belongs. Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof. The disclosures of patents, references and publications cited in the application are incorporated by reference herein.

### EXAMPLES

### Materials

Metal salts: cobalt chloride hexahydrate (CoCl₂·6H₂O), nickel chloride hexahydrate (NiCl₂·6H₂O), manganese chloride tetrahydrate (MnCl₂·4H₂O), copper chloride dihydrate (CuCl₂·2H₂O), rhodium chloride hydrate (RhCl₃·xH₂O), and vanadium chloride (VCl₂), potassium acetate (CH₃COOK), lithium chloride (LiCl₂), magnesium chloride (MgCl₂), potassium carbonate (K₂CO₃) sodium chloride (NaCl), potassium chloride (KCl), potassium sulfate (K₂SO₄), triethyl phosphate (TEP), dimethyl methyl phosphate (DMMP), toluene, n-hexane, dimethyl formamide, methanol, and acetonitrile were purchased from Sigma Aldrich. 2,2-dichlorovinyl dimethyl phosphate (DDVP, also known as dichlorvos) was generously provided by Adama Ltd, Beer Sheva, Israel.

Interdigitated gold electrodes (Dimensions: 10×6 ×0.75 mm; glass substrate; Insulating layer: EPON SU8 resin; electrode material: Au; electrode thickness: 150 nm; microelectrode width: 10 μm, microelectrode gap: 10 μm; number of fingers: 90 pairs) were purchased from MicruX Technologies (Oviedo, Spain).

### Humidity Generation

Different relative humidity (RH) environments were generated by saturated aqueous solutions of lithium chloride (12.3%), magnesium chloride (33.8%), potassium carbonate (43.5%), cobalt chloride (64.2%), sodium chloride (74.4%), potassium chloride (84.7%) and potassium sulfate (98.1%), in airtight glass vessel at a temperature of 25° C.

Relative humidity values designated were also confirmed by a standard humidity and temperature sensor (TH 210, KIMO, Instruments, France).

### Vapor Sensing

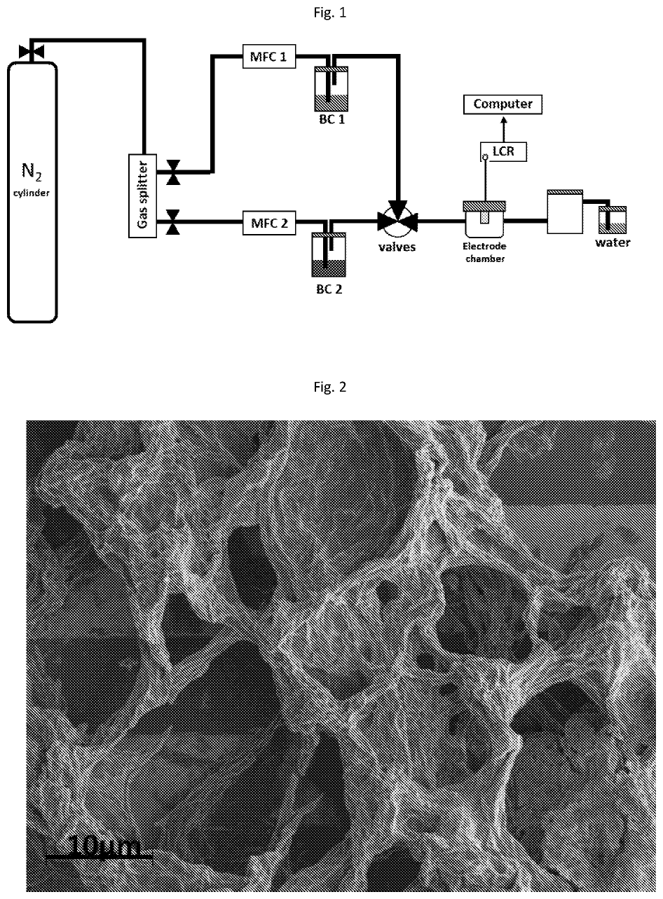

The custom setup, as shown in FIG. 1, consisted of dual-line vapor delivery system and a testing system. Briefly, the carrier flow was split into two components: one carrier flow was used to supply the tested compounds, and the other was used to control the humidity. The tested volatile organic compounds were toluene, dimethylformamide, n-hexane, methanol, and acetonitrile, and the tested organophosphate gases were triethyl phosphate, dichlorovos, and dimethyl methyl phosphate. In the FIG. 1, The custom setup consists of dual-line vapor delivery system and a testing system. In the vapor delivery system, dry nitrogen gas (“N₂ cylinder”) was used as reference carrier and diluting gas. After passing the stainless-steel “gas splitter”, the nitrogen gas real-time flowrate was monitored by two Fathoms Technology mass flow controllers (MFC 1 and MFC 2) manually. The vapor with a standard level was prepared by bubbling the high-purity N₂ gas in a liquid container bubbling chamber (BC) containing liquid organic solvent (in BC1, to create organic vapors) and CoCl₂ saturated salt in water (BC2) to create specific humidity—64% RH. The bubbled gases were mixed (denoted “valves”) and delivered to the testing chamber (“Electrode chamber”), the relative concentrations of the vapors, (in ppmv), was determined using the GC-MS system. The vapor was passed through the electrode chamber, changing the electrode capacitance values that was measured using a “LCR” instrument and the data was collected with the “computer”. The vapors were passed to a larger chamber and then to an open “water” container. The experiments were performed at room temperature (25° C.).

In the vapor delivery system, dry nitrogen gas was used as reference carrier and diluting gas. After passing the stainless-steel gas splitter, the nitrogen gas real-time flow rate was monitored by two Fathoms Technology mass flow controllers (MFC 1 and MFC 2) manually. The vapor with a standard level of tested compounds was prepared by bubbling the high-purity N₂ gas in a liquid container bubbling chamber (BC) containing liquid organic compounds (in BC1, in order to create organic vapors) at variable rates, to produce vapor concentration range was between 5 to 100 ppmv. The second carrier flow was bubbled through CoCl₂ saturated solution in water (BC2) in order to create specific humidity—64% RH. To confirm the retention of constant RH values a standard humidity sensor was used (TH 210, KIMO, Instruments, France). The bubbled gases were mixed and delivered to the testing chamber (not shown in FIG. 1), where the relative concentrations of the vapors was determined using the GC-MS system. The vapor was passed then through the electrode chamber, where changing the electrode capacitance values that was measured using a LCR instrument and the data was collected with the computer. The vapors were passed to a larger chamber and then to an open water container. The experiments were performed at room temperature (25° C.). Vapor selectivity measurements with the volatile organic compounds were carried out at gas concentrations of 30 ppmv, and relative humidity (RH) of 64%. Organophosphate vapor concentration range tested was between 5 to 70 ppmv.

### Capacitance Measurements

All capacitance measurements were carried out manually on LCR meter (Model 878B/879B, BK Precision, USA). The measurements were done at room temperature and under standard conditions upon exposure of the IDE/pGO-Mⁿ⁺ electrodes to the target vapor. The electrode was first saturated at RH 64% using a continuous flow of N₂ gas. Capacitance values where recorded every 1.3 sec, after exposure to 64% RH, producing a clear baseline. Upon addition to different vapor analytes at a specific flow (calibrated to the desired gas concentration following calibration with GC-MS) the change in capacitance was recorded. Capacitance recovery upon the removal of the VOCs and flashing with 64% humidity was carried out after reaching constant saturated capacitance values.

### Instrumental Analyses

### X-Ray Photoelectron Spectroscopy (XPS):

Concentrated solutions of pGO and pGO-Mn⁺ derivatives were placed on silicon wafers and measurements were performed using an X-ray photoelectron spectrometer ESCALAB 250 ultrahigh vacuum (1×10⁻⁹ bar) apparatus with an AlKα X-ray source and a monochromator. The beam diameter was 500 μm with pass energy (PE) of 150 eV for survey spectra and 20 eV for high resolution spectra. The AVANTGE program was used to process the XPS results.

### Fourier Transform-Infrared (FT-IR):

FTIR spectra were recorded on a Nicolet FTIR spectrometer (6700 FTIR spectrometer), using the attenuated total reflectance (ATR) technique with a diamond crystal, collecting data with clean crystal as a background. For each sample, a reference spectrum was first acquired from a clean crystal then the spectra of dry samples were recorded. Analysis was carried out using Omnic (Nicolet, Madison, Wis., USA) software.

### Scanning Electron Microscopy (SEM):

Scanning electron microscopy (SEM) images of the deposited pGO derivatives on IDE samples were acquired on a FEI Verios, SEM (Thermo Fisher Scientific, XHR 460L). The images were viewed at different magnification, at an acceleration voltage of 3 kV.

### Energy-Dispersive X-Ray Spectroscopy (EDS):

The EDS detector with a coincidence point at 4 mm WD, provides X-ray acquisition to obtain high-resolution two-dimensional elemental distribution map throughout the sample surface. Mapping was performed using the AZtec software at an acceleration voltage of 3 kV with 5,000 magnification.

### Thermal Gravimetric Analysis (TGA):

Thermogravimetric analysis (TGA) was carried out using a Q500 TA Instruments (USA). Thermal analysis was performed by heating the samples from 30 C.° to 800 C.° at a heating rate of 10 C°/min under nitrogen flow.

### Raman Spectroscopy:

Raman scattering measurements were performed on a Lab-Ram high-resolution analytical Raman (excitation source was a 633 nm laser and 50×long focal length objective lenses).

### Gas Chromatography-Mass Spectrometry (GC-MS):

Gas Chromatography-Mass Spectrometry was used to detect the analyte concentration at a specific flow rate (controlled with the mass flow controller). The unit's Agilent 7890B GC was connected to an Agilent 5977A single-quadrupole mass-selective detector. The instrument was equipped with a 100-vial autosampler, an NIST02 MS and an ACD Labs MS Manager (software package for mass-spectra interpretation and structure elucidation). Column type of 35% phenyl methyl siloxane for MS, length 30 m, 0.25 mm, I.D. & 0.25 lam film thickness was used. Temperature gradient was programmed from 25 C.° for 1 min, ramping to 70 C.° at 3 C°/min, and then to 280 C.° at 10 C°/min. Transfer line temperature was kept at 280 C°. Total run time was 37 min. The carrier (helium) gas was supplied at flow rate of 2 ml/min. The analytes were quantified based on peak area, using the extracted ion method performed by Masshunter qualitative analysis software. Peak identities were verified by the respective spectra from Masshunter MS library.

Samples were supplied from solution (for standards calibration purposes), by taking 1 μl of standards' solutions. A 20 μl syringe was used for collecting the analyte vapor sample, injecting directly to the GC-MS (spitless). High purity solvents were used in order to prepare the standard solutions (TEP, DMMP, toluene, n-hexane, dimethyl formamide, methanol, and acetonitrile with 99% purity, DDVP and DMMP with 97% purity; all standards were prepared in methanol solution, except of the methanol standard which was prepared in acetonitrile. For each analyte a calibration curve with a known concentration (5 ppm-1000 ppm) was prepared. The flow rates for the capacitance measurements were adjusted to produce 30 ppmv gas concentrations for each examined volatile organic analyte, and to the desired testing concentration for the organophosphate compounds.

### EXAMPLE 1—Fabrication of Composite Metal-Graphene Oxide Sensors

Pristine graphite oxide was synthesized from graphite powder using a modified Hummer's method (Zhao et al. ACS Nano 2010, 4, 5245). The graphite oxide was re-dissolved in double distilled water (10 mg/mL) to obtain a graphite oxide solution which was ultra-sonicated for 1 hour to obtain a stable graphene oxide (GO) suspension.

The composites GO-Mⁿ⁺ were synthesized through mixing of aqueous graphene oxide suspension (2 mg/mL) and aqueous metal-salt solutions (Co²⁺, Ni²⁺, Mn²⁺, Cu²⁺, V²⁺, Rh³⁺; all at 10 mM). The final concentrations of the mixtures were maintained at 1 mg/mL graphene oxide suspension and 5 mM metal-salt solution. The mixture solutions (GO-Mⁿ⁺) were kept for two hours to maximize the interaction between GO and the metal ions. The mixtures were subsequently centrifuged and washed with water to remove non-bonded metal ions, and dried at 70° C. for 12 hours. The GO-Mⁿ⁺ composites were re-dissolved in ultrapure distilled water (18.3 mΩ, Millipore) at concentrations of 1 mg/mL, and sonicated for 1 hour to make the suspension homogeneous.

To prepare the pGO-Mⁿ⁺ capacitive electrodes, briefly, GO-Mⁿ⁺ suspensions were drop-cast (10 μL), on the interdigitated electrodes (IDEs) and retained thereon, allowing it to dry slowly, at room temperature for one hour. The obtained assemblies, GO-IDE, GO-Co²⁺-IDE, GO-Ni²⁺-IDE, GO-Mn²⁺-IDE, GO-Cu²⁺-IDE, GO-V²⁺-IDE, and GO-Rh³⁺-IDE, were placed in 4 ml glass vials, deep-frozen in liquid nitrogen for 3 minutes, and lyophilized for 24 h to remove the remaining water and to obtain the porous GO film on the IDEs (pGO/IDES). The resultant electrodes—pGO, pGO-Co²⁺, pGO-Ni²⁺, pGO-Mn²⁺, pGO-Cu²⁺, pGO-V²⁺ and pGO-Rh³⁺—were used in the capacitive based chemical vapor sensing applications. Three separate electrodes were employed in each experiment.

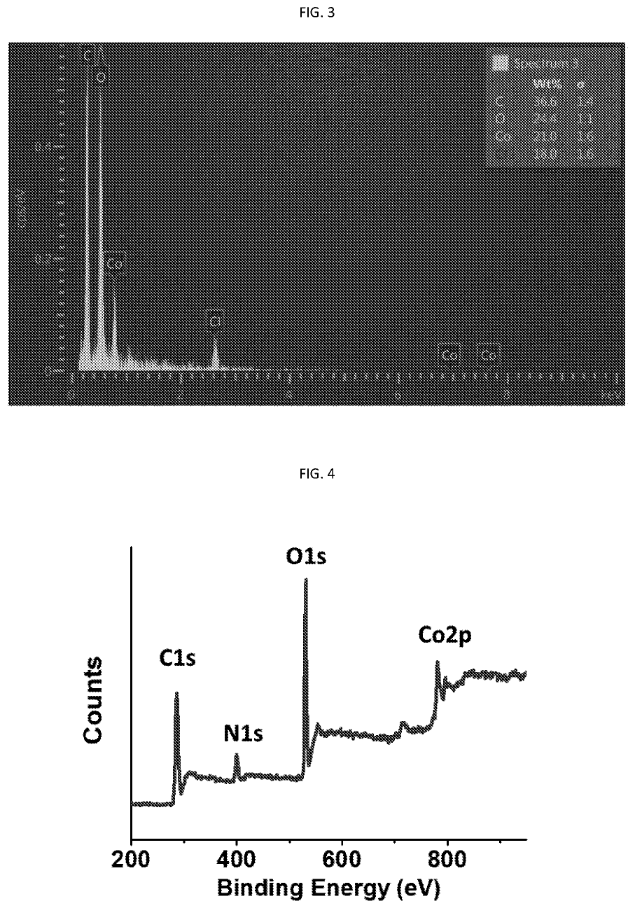

A representative scanning electron microscopy (SEM) image, of pGO-Co²⁺, is presented in FIG. 2. In the image, with the scale bar indicating the size of 10 micrometers, it can be readily discerned that pGO domains are attached onto the IDE surface (the horizontal gold “fingers” are clearly shown in the image), demonstrating the substantial surface area available for gas adsorption. The image was viewed at 2000× magnification, at an acceleration voltage of 3 kV. Turning now to FIG. 3, results of energy dispersive x-ray spectroscopy (EDS) analysis are presented, which was carried out in conjunction with the SEM experiment, on a representative area of approximately 4×2.5 micrometers. As it can be readily seen from the graph, the cobalt ions associated with the pGO framework are ubiquitously present. The EDS spectrum of pGO-Co²⁺, exhibiting a strong carbon and oxygen peaks which agree with the high percentage of carbon and oxygen in GO. The EDS spectra also revealed medium cobalt peak confirming that the pGO-Co²⁺ have been successfully prepared.

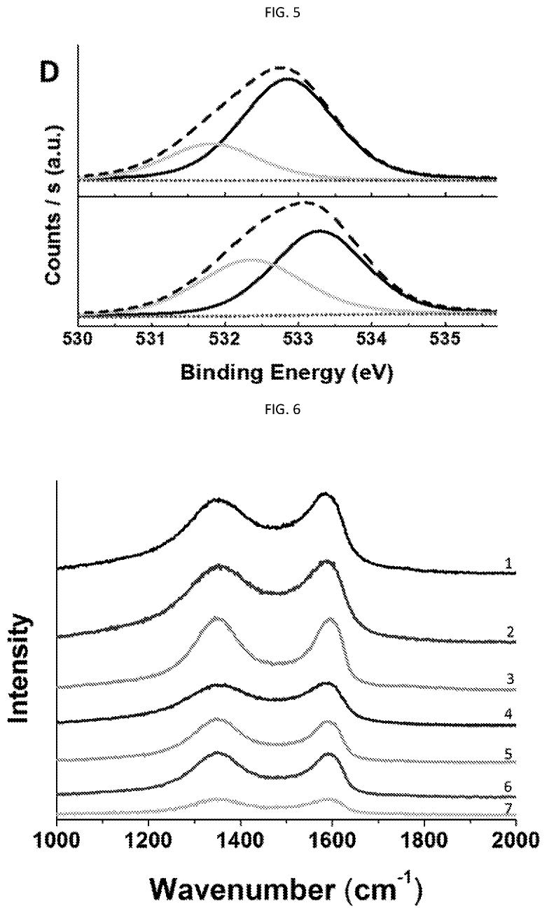

Referring now to FIG. 4 and FIG. 5, wherein the x-ray photoelectron spectroscopy (XPS) data is presented. In the FIG. 4, concentrated solutions of pGO and pGO-Co²⁺ derivatives were placed on silicon wafers, and measurements were performed using an ESCALAB 250 X-ray photoelectron spectrometer ultrahigh vacuum (1×10⁻⁹ bar) apparatus with an AlKα X-ray source and a monochromator. The beam diameter was 500 lam with a pass energy (PE) of 150 eV for survey spectra and 20 eV for high resolution spectra. The AVANTGE program was used to process the X-ray photoelectron spectroscopy (XPS) results. O1 s XPS of pGO (upper spectrum) and pGO-Co²⁺ (lower spectrum) are depicted. The black dash spectrum corresponds to the experimentally recorded result, while the solid black and grey spectra represent the deconvoluted peaks of C—OH and C═O, respectively.

The XPS survey is shown in the FIG. 4, while the high-resolution O 1 s XPS of pGO (upper spectrum) and pGO-Co²⁺ (lower spectrum) are depicted in the FIG. 5; the dashed spectrum corresponds to the experimentally-recorded result, while the earlier lower peak spectrum and the later higher peak spectrum represent the de-convoluted peaks of C—OH and C═O, respectively. The spectra confirm incorporation of cobalt within the pGO matrix, and indicate binding of the metal ions to oxygen-containing moieties in the pGO framework. In particular, the O-1 s spectrum (FIG. 5) reveals changes in peak positions and intensities following incorporation of the cobalt ions. Specifically, the deconvoluted peak at 533.3 eV, ascribed to C—OH units, shifted to a lower binding energy (532.8 eV) and became more intense upon Co²⁺ addition, while the peak corresponding to C═O residues, at 532.4 eV, became significantly less intense upon Co²⁺ binding. The energy shifts and intensity modulation of the O-1 s peaks probably reflect changes in electron densities around the oxygen atoms upon formation of coordinative bonds with the Co²⁺ ions. The XPS data allowed quantification of the metal ions in the composite porous graphene oxide. Thus, pGO-Co²⁺ contained about 8.75% of cobalt, pGO-Ni²⁺ contained about 7.69% of nickel, pGO-Mn²⁺ contained about 1.87% of manganese, pGO-Co²⁺contained about 8.75% of cobalt, pGO-Cu²⁺ contained about 2.41% of copper, pGO-Rh³⁺ contained about 6.34% of ruthenium, and pGO-V²⁺ contained about 6.02% of vanadium.

Further, the sensors were tested using RAMAN spectroscopy, and the results are shown in the FIG. 6. In the figure, two dominant peaks around 1350 and 1585 cm⁻¹ can be observed, corresponding to the D and G band of graphitic sp2 bond. The numbers represent the tested specimens, as follows: (1) pGOx-Co²⁺, (2) pGOx-Ni²⁺, (3) pGO, (4) pGOx-Mn²⁺, (5) pGOx-Cu²⁺, (6) pGOx-V²⁺ and (7) pGOx-Rh³⁺. It can be readily observed that the ratio between the ID Raman peak (area under the Raman signal appearing at 1350 cm⁻¹) and IG peak (at around 1575 cm⁻¹) is significantly lower for pGO-Co²⁺ and pGO-Ni²⁺ as compared to electrodes comprising other metal ions. The ID/IG ratio usually reflects the degree of planar organization in comparison to structural defects in nanocrystalline carbon materials, particularly graphene oxide. Specifically, while low ID/IG ratios account for high concentrations of carbon atoms adopting sp2 coordination in planar environments, an increase in the ID/IG ratio may indicate greater abundance of defects and/or amorphous GO structures. The ID/IG ratios of the sensors are presented in the table 1 below, alongside with the dielectric constant (as F/m), calculated from the capacitance measurement, according to the following formula:

$$C = {ηε}_{o} {ε}_{r} \frac{lt}{d}$$

in which C is capacitance in farads (F), η is the number of fingers of interdigital electrode, ε₀ is the permittivity of free space (ε₀=8.854×10⁻¹² F/m), εᵣ is the relative permittivity, commonly known as the dielectric constant, 1 is the length of interdigital electrodes, t is the thickness of interdigital electrodes and d is the distance between the electrodes.

TABLE 1

| | pGO- | pGO- | | pGO- | pGO- | pGO- | pGO- |
|---|---|---|---|---|---|---|---|
| | Co²⁺ | Ni²⁺ | pGO | V²⁺ | Rh³⁺ | Cu²⁺ | Mn²⁺ |
| εᵣ | 1570.0 | 2160.0 | 153.1 | 33.6 | 24.1 | 2.2 | 1.8 |
| ID/IG | 1.52 | 1.49 | 1.82 | 1.93 | 2.00 | 2.17 | 2.14 |

Notably, pGO-Co²⁺ and pGO-Ni²⁺ exhibited smaller ID/IG ratios than the parent pGO material attesting to the significant structural effect they exerted following incorporation within the pGO matrix. In contrast, association of other metal ions with pGO gave rise to lesser abundance of sp2 carbon atoms within the pGO framework, generating defect carbon sites and the resultant higher ID/IG ratios. These structural transformations likely account for the lower dielectric constants of pGO associated with metal ions other than Co²⁺ and Ni²⁺.

Thermogravimetric analysis of the pGO-Miⁿ⁺ was also conducted to assess the amount of bound water. The TGA data reveal that significant concentrations of [metal-coordinated] water molecules were immobilized within the pGO-Co²⁺ and pGO-Ni²⁺ frameworks, much less so in case of pGO associated with other metal ions. The weight loss of the sensors, attributable to loss of metal-coordinated water (up to 125° C.), are depicted in Table 2 below. Specifically, the TGA trace of pGO-Mⁿ⁺ shows an initial weight loss of approximately 20% at around 110 C.° due to evaporation of the embedded metal-coordinated water molecules, while the subsequent weight decrease of ˜30% occurring at about 210 C.° is attributed to decomposition of oxygen-containing functional groups within pGO.

TABLE 2

| pGO-Mⁿ⁺ type | Weight loss (%) |
|---|---|
| pGO-Co²⁺ | 19.80% |
| pGO-Ni²⁺ | 18.69% |
| pGO-Mn²⁺ | 15.53% |
| pGO-Cu²⁺ | 8.97% |
| pGO-V²⁺ | 7.73% |
| pGO-Rh³⁺ | 11.72% |

Further, pGO-Co²⁺ and pGO-V²⁺ were analyzed by Fourier transform infrared (FTIR) spectroscopy. Vanadium composite was chosen as representative of low-dielectric constant material, which has also not demonstrated capacitive response to TEP and other OP tested, vide infra. The FTIR spectra in the region between 1450-1850 cm⁻¹ corresponding to C═C and C═O vibrations demonstrated more pronounced shift of pGO C═O stretch vibration at around 1720 cm⁻¹ to lower frequencies in case of pGO-Co²⁺, which can likely be ascribed to the pronounced oxygen coordination with the Co²⁺ ions, resulting in lower electron densities around the GO framework oxygen atoms and corresponding reduced C═O bond stiffness. Such interactions are much less significant in case of pGO-V²⁺ accounting for the insignificant spectra shift —consistent with the Raman data. Indeed, the broadening of the C═C peak of pGO at around 1570 cm⁻¹ upon embedding V²⁺ is consistent with the greater abundance of structural defects in the pGO framework upon addition of the vanadium ions. The O—H stretch region at between 2750 cm⁻¹-3150 cm⁻¹ further attests to the significantly divergent structural impact of Co²⁺ vs V²⁺ incorporation within the pGO matrix. Specifically, in case of pGO-V²⁺, the pronounced decrease in the intensity of 0-H vibration at 3400 cm⁻¹ relative to pGO, can be attributed to elimination of oxygen units upon ion-induced reduction and concomitant increase in defect sites within the pGO matrix. In contrast, the intensity of the 0-H peak was not attenuated in case of pGO-Co²⁺, indicative of retaining the graphitic sheet organization upon incorporation of the cobalt ions.

The FTIR spectra were also obtained in presence of TEP. In the 0-H stretch region recorded after addition of TEP to the pGO-metal assemblies further structural differences between pGO-Co²⁺ and pGO-V²⁺ are revealed, likely accounting for the distinct capacitive responses of the two materials. Specifically, the addition of TEP to pGO-Co²⁺ gave rise to elimination of the shoulder at around 3600 cm⁻¹, reflecting the replacement of the corresponding population of “network water” (i.e. poorly connected water molecules) with the TEP ligands, which was not observed with vanadium pGO.

### EXAMPLE 2—Sensing Organophosphate Gases with Metal-Composite Capacitive Sensors

The electrodes comprising pGO coupled to different metal ions as described above were exposed for few minutes to vapors (each gas at a concentration of 30 ppmv) of various analytes. The capacitance responses of the pGO-Co²⁺ electrode and other sensors to these gases are summarized in table below, demonstrating remarkable sensitivity and selectivity for organophosphates of the sensors couples with cobalt and nickel ions. As apparent in tables 3 and 4 below, pGO-Co²⁺ and pGO-Ni²⁺ exhibited selectivity for organophosphate gases [TEP; 2,2-dichlorovinyl dimethyl phosphate (DDVP, commonly known as dichlovos); dimethyl methyl phosphate (DMMP)] compared to other tested vapors. Furthermore, as readily seen from the Table 3, remarkable sensitivity was apparent, particularly in case of pGO-Co²⁺, which featured, for example, a capacitance response of ˜340 in case of TEP.

TABLE 3

| | TEP | | DDVP | | DMMP | |
|---|---|---|---|---|---|---|
| Derivatives | C₀/C_{gas} | Error | C₀/C_{gas} | Error | C₀/C_{gas} | Error |
| pGO-Co²⁺ | 341.00 | 32.00 | 181.00 | 25.00 | 221.00 | 35.80 |
| pGO-Ni²⁺ | 63.23 | 10.2 | 23.52 | 12.44 | 39.90 | 14.00 |
| pGO | 1.48 | 0.20 | 1.00 | 0.19 | 14.00 | 6.00 |
| pGO-Mn²⁺ | 1.76 | 0.08 | 2.00 | 0.43 | 2.00 | 0.10 |
| pGO-Cu²⁺ | 1.70 | 0.39 | 2.86 | 0.66 | 1.03 | 0.40 |
| pGO-V²⁺ | 9.00 | 0.57 | 15.00 | 3.00 | 1.10 | 0.33 |
| pGO-Rh3⁺ | 1.82 | 0.22 | 1.81 | 0.12 | 1.83 | 0.64 |

TABLE 4

| | ACN | | Methanol | | DMF | | Toluene | | Hexane | |
|---|---|---|---|---|---|---|---|---|---|---|
| Derivatives | C₀/C_{gas} | Error | C₀/C_{gas} | Error | C₀/C_{gas} | Error | C₀/C_{gas} | Error | C₀/C_{gas} | Error |
| pGO-Co²⁺ | 1.10 | 0.30 | 1.17 | 0.30 | 3.19 | 0.48 | 11.40 | 2.11 | 1.89 | 0.10 |
| pGO-Ni²⁺ | 1.82 | 0.06 | 3.08 | 0.15 | 3.15 | 0.33 | 10.00 | 1.06 | 2.21 | 0.24 |
| pGO | 1.27 | 0.17 | 1.85 | 0.12 | 1.16 | 0.14 | 1.16 | 0.09 | 1.13 | 0.03 |
| pGO-Mn²⁺ | 1.27 | 0.06 | 2.01 | 0.10 | 2.00 | 0.52 | 1.64 | 0.13 | 1.14 | 0.10 |
| pGO-Cu²⁺ | 1.74 | 0.10 | 1.50 | 0.42 | 5.57 | 0.32 | 1.88 | 0.15 | 1.00 | 0.00 |
| pGO-V²⁺ | 1.35 | 0.06 | 1.96 | 0.16 | 10.66 | 0.66 | 1.08 | 0.02 | 3.17 | 0.19 |
| pGO-Rh3⁺ | 1.33 | 0.10 | 1.45 | 0.30 | 1.33 | 0.10 | 1.48 | 0.26 | 1.19 | 0.32 |

### EXAMPLE 3—Sensing Organophosphate Gases with pGO-Co²⁺ Capacitive Sensors

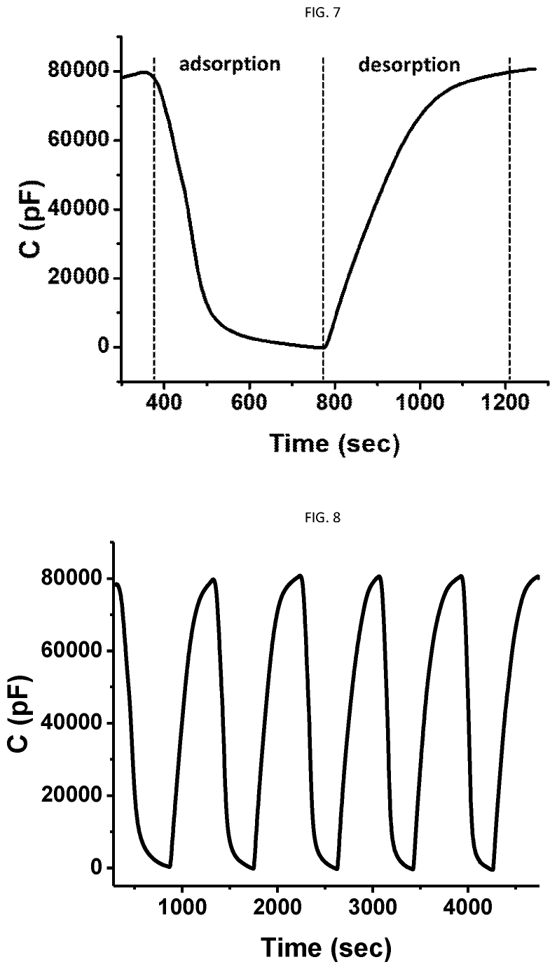

The pGO-Co²⁺ sensors were exposed to triethyl phosphate at varying concentrations. The response of pGO-Co²⁺ sensor upon exposure to of 30 ppmv triethyl phosphate (TEP), a representative organophosphate gas, is shown in FIG. 7 to FIG. 9.

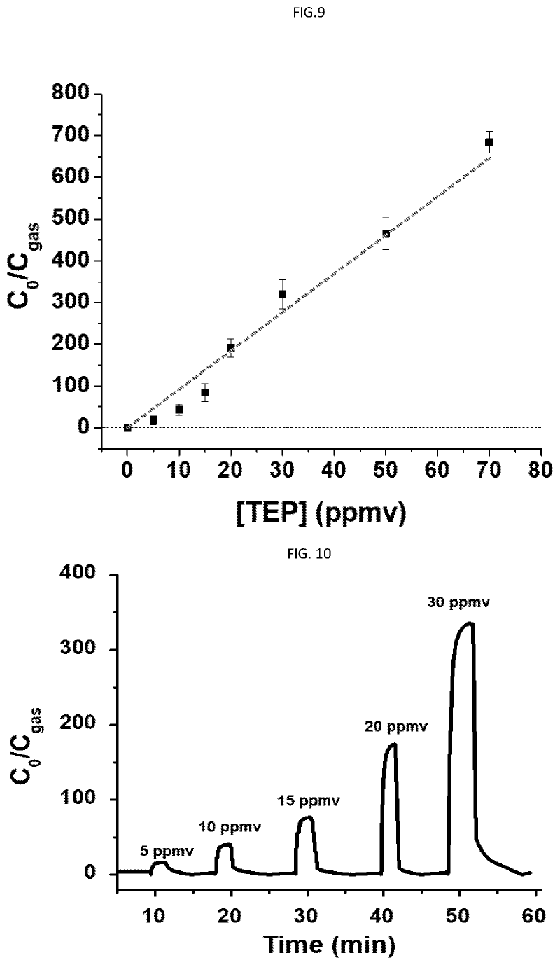

Therein, graphs of capacitance response change (denoted as “C₀/C_{gas}”) is plotted versus time of exposure (FIGS. 7 and 8), or versus the concentration of TEP (denoted “[TEP] (ppmv)”, FIG. 9). FIG. 7 demonstrates capacitive signals recorded upon adsorption and desorption of TEP at a concentration of 30 ppmv, FIG. 8—TEP adsorption/desorption cycles, and FIG. 9 TEP dose-response curve in the range of 0-70 ppmv concentration, with the dashed line corresponds to the linear regression with R²=0.98, and top dash line corresponding to y=0. In the presence of TEP the capacitance decreased drastically, with complete response time of the pGO-Co²⁺ sensor upon TEP addition of about 150 sec, with a significant change being observed almost instantaneously. Following evacuation of the TEP gas, the capacitance gradually increased, reaching the initial baseline within 5 minutes.

As demonstrated in the table 5 below, this response time is better than many other organophosphate vapor sensors, and reflects the fast adsorption kinetics of TEP vapor molecules onto the pGO-Co²⁺ matrix.

TABLE 5

| | | Threshold | Response | Recovery |
|---|---|---|---|---|
| Reference | Working principle | detection | time | time |
| Wang, Y.; et al, Journal | Resistive reduced | DMMP: dimethyl | 3 min | 4 min |
| of Materials Chemistry C | graphene oxide | methylphosphonate | | |
| 2019, 7 (30), 9248-9256. | multilayer network | 1-50 ppm | | |
| | | 2.21-8.95 | | |
| | | Response (%) | | |
| Hang, C. P.; Yuan, C. L. | Resistance Changes in | DMMP | 4 min | 12 min |
| Journal of Materials | MWNTs-PANI film | 332 ppm | | |
| Science 2009, 44 (20), | caused by changes in | 12 Response(%) | | |
| 5485-5493. | interlayers distance | | | |
| K. Cattanach, et al, | Resistance Changes in | DMMP | 20 min | 10 min |
| Nanotechnology, 2006, 17, | SWNT/PET films caused | 25-50 ppm | | |
| 4123-4128. | by chemisorption on | 5-8 Response(%) | | |
| | SWNT. | | | |
| N. T. Hu, et al | Resistance Changes in | DMMP | 18 min | 6 min |
| Sens. Actuators, B, 163 | p-phenylenediamine | 5-80 ppm | | |
| (2012), pp. 107-114 | reduced graphene | 5-14 Response(%) | | |
| | oxide caused by | | | |
| | chemisorption. | | | |

### EXAMPLE 4—Stability of pGO-Co²⁺ Capacitive Sensors

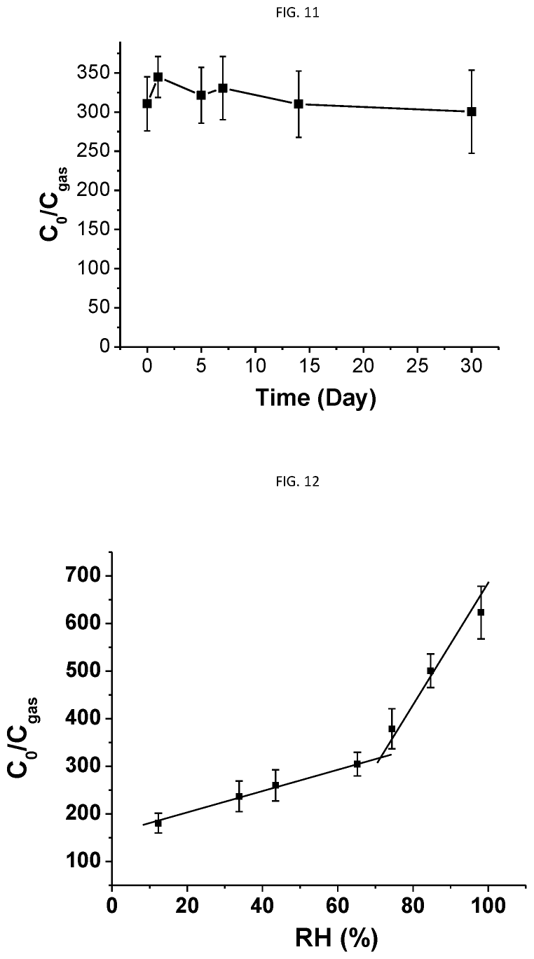

The pGO-Co²⁺ sensors were subjected to cycles of exposure to TEP at 30 ppmv, and washout with nitrogen at 64% RH. The results have demonstrated excellent response/recovery repeatability of the pGO-Co²⁺ sensors, as shown in FIG. 8. The calibration curve in FIG. 9 further attests to the outstanding performance of the pGO-Co²⁺ sensor, showing a linear relationship between capacitance response and TEP concentrations. The sensitivity threshold of 5 ppmv, apparent in FIG. 9, is very low, and the dynamic range of ˜700 in the linear response regime underscores the extraordinary sensitivity of the pGO-Co²⁺ sensor. The raw capacitive response curves of pGO-Co²⁺ to TEP vapors between concentrations of 5-30 ppmv are presented in FIG. 10. The electrode displayed excellent stability and repeatability, as seen in FIG. 11, wherein capacitive response signals recorded upon adsorption and desorption of TEP at a concentration of 30 ppmv to triethyl-phosphate vapor. Triplicates of pGO-Co²⁺ electrode were tested at several time points from the day of preparation (o day), and after 1, 5, 7, 14 and 30 days. Shown are mean values with calculates standard deviation. It can be readily seen that even after 30 days capacitance result exhibits excellent stability and repeatability. All electrodes were kept under the same temperature conditions in N₂ environment.

Further, the capacitance response of pGO-CO²⁺ at different relative humidity values was tested, and the results are demonstrated at FIG. 12. It can be readily seen that although the sensors do react to the changes in the relative humidity, particularly to extreme values, the capacitive signals to OPs are significant. There are two linear windows in the capacitance response to water vapor versus dry nitrogen, one between 12.3 and 64.2%, while another is between 74.4 and 98.1%. The separation to two linear domains is possibly due to the different adsorption mechanisms of water molecules on the pGO-Co²⁺ surface, ascribed to the transformation between monolayer chemisorption and multilayer physisorption, previously observed in the case of pGO.

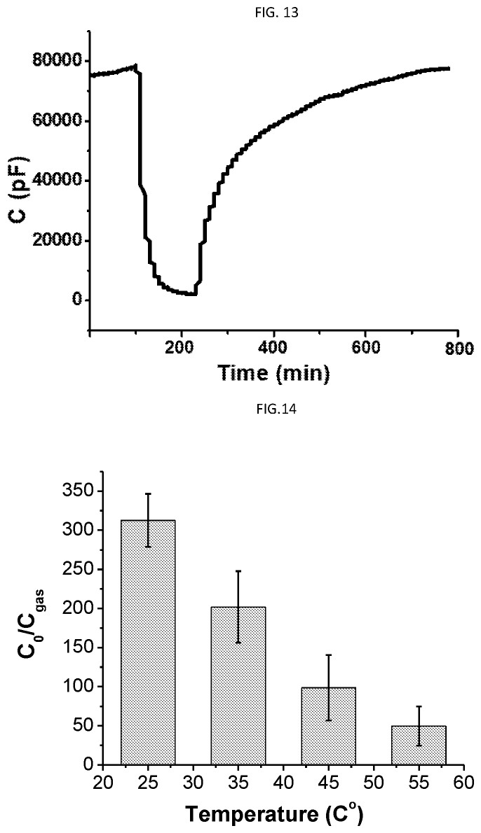

When air was used as a carrier gas for TEP, the capacitive response of pGO-Co²⁺ to triethyl-phosphate vapor was slightly lower, but not longer, as attested by FIG. 13. However, a significant decline in the response was observed at elevated temperatures, between 25° C. and 55° C., as can be seen in FIG. 14, where the temperature was controlled by an outer heating source to the electrode chamber, although the lowest capacitance response was still around 50 at 55° C.

### EXAMPLE 5—Sensing Organophosphate Gases in Gas Mixtures with pGO-Co²⁺ Capacitive Sensors

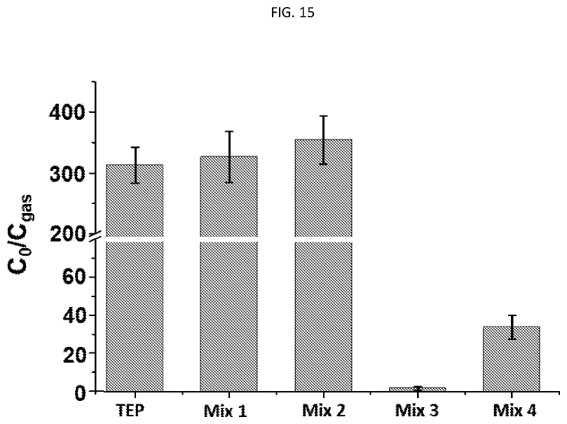

Various gas mixtures were prepared, with the concentrations determined by GC-MS. Mix 1 consisted of TEP (35±5 ppmv), acetonitrile (50±5 ppmv), and hexane (55±5 ppmv); mix 2 consisted of TEP (35±5 ppmv), methanol (30±5 ppmv), and toluene (35±5 ppmv); mix 3 consisted of acetonitrile (50±5 ppmv) and hexane (55±5 ppmv); mix 4 consisted methanol (30±5 ppmv), and toluene (35±5 ppmv). Capacitive response, on the inverted scale, of pGO-Co²⁺ towards different vapor mixtures is demonstrated in FIG. 11. The representative bar diagram reveals, that significant capacitive signals were retained upon exposure of the sensor to TEP, even when mixed with other polar and non-polar gases. Importantly, this confirms that the capacitance changes in the mixtures were directly related to the presence and the concentration of TEP. The data are summarized in FIG. 15.

1-33. (canceled)

34. A capacitive sensor for detection of organophosphate vapors, said sensor comprising dielectric material integrally formed on a pair of electrodes, said dielectric material comprising transition-metal composite of porous graphene oxide.

35. The sensor according to claim 34, wherein said composite comprises between 5 and 12 weight percent of a transition metal, and optionally wherein said composite comprises between 7 and 9 weight percent of a transition metal.

36. The sensor according to claim 34, wherein said transition metal in said composite is selected such that the dielectric constant of said dielectric material is above 150 F/m, and optionally wherein said dielectric constant is above 1000 F/m.

37. The sensor according to claim 34, wherein said transition metal in said composite is selected such that in the composite a ratio between the area under the Raman signal appearing at ˜1350 cm-1 and the area under the Raman signal appearing at ˜1575 cm-1 is between 1 and 1.9.

38. The sensor according to claim 34 wherein said dielectric material comprises between 8 and 25 weight percent of adsorbed water, and optionally wherein said dielectric material comprises between 14 and 22 weight percent of adsorbed water.

39. The sensor according to claim 34, wherein said transition metal is selected from the group consisting of cobalt, nickel, titanium, ruthenium, palladium, and zirconium, and optionally wherein said transition metal is present in a form of a cation, and optionally wherein said transition metal is in a form of Co2+ or Ni2+.

40. The sensor according to claim 34, wherein said pair of electrodes is in form of interdigitated electrodes.

41. A sensing device for the detection of organophosphates vapors in the air, said device comprising a capacitive sensor according to any one of the preceding claims.

42. The device according to the claim 41, further comprising a temperature controlling unit, and optionally wherein said temperature controlling unit is in thermal connection with said capacitive sensor.

43. The device according to claim 41, further comprising a humidity compensation sensor.

44. The device according to claim 41, wherein said capacitive sensor being conductively connected to an electrical circuit adapted to monitor the capacitance of the sensor.

45. The device according to claim 41, further comprising an effector sub-circuit configured to produce a notification upon a change in the capacitance of said sensor, indicative of the presence of an organophosphate vapor, and optionally wherein said notification is in a form of an alarm sound, in a form of deflection of a pointer, or in a form of an electromagnetic signal.

46. The device according to claim 41, comprising a plurality of said capacitive sensors in form of an array.

47. A process of manufacturing a sensor as claimed in claim 34, said process comprising providing a pair of electrode and integrally forming thereon a coating comprising metal-composite porous graphene oxide.

48. The process according to claim 47, wherein said metal-composite porous graphene oxide comprises cobalt or nickel, and optionally wherein a weight ratio between said metal and said graphene oxide is between 5 and 12 weight percent.

49. The process according to claim 48, comprising providing a metal-composite graphene oxide precursor liquid, by combining in an aqueous medium a metal source and a graphene oxide dispersion, and optionally wherein said metal precursor in an inorganic salt of said metal.

50. The process according to claim 49, wherein a weight ratio between said metal and said graphene oxide is between 0.3:1 and 1:1, in said precursor liquid.

51. The process according to claim 50, further comprising purifying said metal-composite porous graphene oxide precursor liquid, by separating said metal-composite graphene precursor and said aqueous medium, and resuspending said separated metal-composite porous graphene oxide precursor in water.

52. The process according to claim 50, further comprising applying said metal-composite porous graphene oxide precursor liquid onto said pair of electrodes, and optionally wherein said applying is performed at a temperature ranging from 10□C to 60□C, and further optionally wherein said applying is performed for an incubation time of at least 5 minutes, and optionally wherein said incubation time is between 45 and 75 minutes.

53. The process according to claim 50, further comprising freeze-drying said metal-composite porous graphene oxide precursor liquid on said electrode, and optionally wherein the amount of water in said precursor liquid after said incubation time and before said freeze-drying is between 25% and 40%.
